(19) United States
(12) Patent Application Publication
(10) Pub. No.: US 2023/0326625 A1
(43) Pub. Date: Oct. 12, 2023

(54) HEAT RESISTANT ELECTROCARDIOGRAPH CABLE

(71) Applicant: Ivy Biomedical Systems, Inc., Branford, CT (US)

(72) Inventors: Ryan MCGUIRE, Orange, CT (US); Joseph LANE, Branford, CT (US)

(21) Appl. No.: 17/715,275

(22) Filed: Apr. 7, 2022

(51) Int. Cl. H01B 7/18; H01B 7/02; H01B 7/06; A61B 5/308; H01B 7/22

(52) U.S. Cl. CPC H01B 7/1855; H01B 7/02; H01B 7/06; A61B 5/308; H01B 7/226; A61B 2562/222

(57) ABSTRACT

The present disclosure provides an electrocardiograph cable for use in an MRI system. The electrocardiograph cable includes a cable jacket and a plurality of lead wires extending through the cable jacket. Each of the plurality of lead wires includes a first end segment coupled to a monitoring electrode, a second end segment coupled to a monitoring device, an electrically insulating core extending from the first end segment to the second end segment, an electrically conductive wire having a plurality of turns wound around the electrically insulating core from the first end segment to the second end segment, and an electrically insulating sleeve covering the electrically conductive wire wound around the electrically insulating core.

### BACKGROUND

### Field

The present disclosure relates to electrocardiograph cables, particularly electrocardiograph cables used for monitoring devices in a magnetic resonance imaging (MRI) system.

### Background

MRI systems have been routinely used in the medical field to capture anatomic images of a patient's tissues and organs. Typical equipment for an MRI system includes an electromagnetic scanner for generating a strong magnetic field, for example on the order of 1.5 Tesla or greater, and for applying radio frequency (RF) pulses at a patient, who is usually placed on a table surrounded by the MRI scanner. The MRI scanner usually applies rapidly changing magnetic gradients that vary linearly over space, which allows processing equipment to selectively capture slice imaging of the patient's tissues and organs. An MRI system typically includes a monitoring room shielded from the MRI scanner, where an operating console can receive and process signals transmitted from the sensors of the MRI scanner to generate anatomic images of the patient.

The physiological state of the patient is commonly monitored during the MRI procedure, during which the patient's physiological data is transmitted from monitoring equipment to the operating console. For example, electrocardiogram signals of the patient are monitored by placing electrodes on the patient's torso and electrically connecting the electrodes to a patient monitor via lead wires. The patient monitor collects and transmits electrocardiogram signals to the operating console.

However, the lead wires of the electrocardiograph monitoring unit are typically exposed to the dynamic magnetic gradients and pulsed RF waves applied by the MRI scanner. This exposure can induce significant temperature rises in the lead wires (e.g., 31° C. or greater) that could harm or burn the exposed skin of the patient, ultimately jeopardizing the patient's safety. For example, MRI scanners usually excite the generated magnetic field with 50 kW of RF power, thereby creating a field strength exceeding 1500 V/M. Moreover, the moving magnetic field gradients can induce generation of currents in any exposed conductive material. These rapidly changing magnetic gradients and powerful RF fields can induce eddy currents in the conducting material of the lead wires, which will then heat the lead wire, often enough to cause third degree burns.

Thus, there is a need for an improved lead wire in an electrocardiograph cable that can reduce heating typically induced by RF pulses emitted during an MRI procedure.

### BRIEF SUMMARY

The present disclosure includes various embodiments of an electrocardiograph cable for reducing heat induced by pulsating RF waves applied by an MRI scanner. In some embodiments, the electrocardiograph cable includes a cable jacket. In some embodiments, the electrocardiograph cable includes a plurality of lead wires extending through the cable jacket. In some embodiments, each of the plurality of lead wires includes a first end segment configured to be coupled to a monitoring electrode, a second end segment configured to be coupled to a monitoring device, an electrically insulating core extending from the first end segment to the second end segment, an electrically conductive wire having a plurality of turns wound around the electrically insulating core from the first end segment to the second end segment, and an electrically insulating sleeve covering the electrically conductive wire wound around the electrically insulating core. In some embodiments, a pitch between adjacent turns of the electrically conductive wire remains substantially uniform along a longitudinal axis of the electrically insulating core. In some embodiments, the pitch between adjacent turns of the electrically conductive wire is in a range from about 0.0020 inches to about 0.0001 inches.

In some embodiments, the electrically conductive wire includes a metal alloy-based material having a resistance in a range from about 650 ohm-cir-mil/foot to about 850 ohm-cir-mil/foot.

In some embodiments, the metal alloy-based material of the electrically conductive wire is non-magnetic.

In some embodiments, the electrically conductive wire includes a diameter in a range from about 0.00176 inches to about 0.00250 inches.

In some embodiments, the electrically insulating core includes a glass fiber-based material.

In some embodiments, the electrically insulating core includes a diameter in a range from about 0.250 inches to about 0.035 inches.

In some embodiments, each of the lead wires is configured to maintain an outer surface of the electrically insulating sleeve at a temperature in a range from about 26° C. to about 15° C. when subjected to a time-varying magnetic field having a magnetic flux density in a range from about 3 Tesla to about 10 Tesla.

In some embodiments, the electrically insulating sleeve includes an elastomer-based material.

In some embodiments, each of the lead wires has a distributed resistance of about 10,000 ohms/foot.

In some embodiments, the plurality of lead wires are twisted together along an internal passage of the cable jacket.

The present disclosure includes various embodiments of lead wire in an electrocardiograph cable for reducing heat induced by pulsating radio frequency waves applied by an MRI scanner. In some embodiments, the lead wire includes a first end segment configured to be coupled to a monitoring electrode, a second end segment configured to be coupled to a monitoring device, an electrically insulating core extending from the first end segment to the second end segment, an electrically conductive wire having a plurality of turns wound around the electrically insulating core from the first end segment to the second end segment, and an electrically insulating sleeve covering the electrically conductive wire wound around the electrically insulating core. In some embodiments, the electrically insulating core includes a diameter in a range from about 0.250 inches to about 0.035 inches. In some embodiments, the electrically conductive wire includes a heat capacity configured to maintain an outer surface of the electrically insulating sleeve at a temperature in a range from about 26° C. to about 15° C. when subjected to a time-varying magnetic field having a magnetic flux density in a range from about 3 Tesla to about 10 Tesla.

The present disclosure includes various embodiments of a method controlling a temperature of a lead wire in an electrocardiograph cable during an MRI procedure. In some embodiments, the method includes a step of providing an electrically insulating core of the lead wire with a diameter in a range from about 0.250 inches to about 0.035 inches. In some embodiments, the method includes a step of providing an electrically conductive wire of the lead wire with a resistance in a range from 650 ohm-cir-mil/foot to about 850 ohm-cir-mil/foot. In some embodiments, the method includes a step of winding the electrically conductive wire into a plurality of turns around the electrically insulating core from a first end segment of the lead wire to a second end segment of the lead wire. In some embodiments, the method includes a step of maintaining a pitch in a range from about 0.0020 inches to about 0.0001 inches between adjacent turns of the electrically conductive wire uniformly along a longitudinal axis of the electrically insulating core. In some embodiments, the method includes a step of covering the electrically conductive wire wound around the electrically insulating core with an electrically insulating sleeve of the lead wire. In some embodiments, the method includes a step of subjecting the lead wire to pulsating radio frequency waves and a time-varying magnetic field having a magnetic flux density in a range from about 3 Tesla to about 10 Tesla. In some embodiments, the method includes a step of dissipating heat away from the electrically conductive wire to maintain an outer surface of the electrically insulating sleeve at a temperature in a range from about 26° C. to about 15° C.

### BRIEF DESCRIPTION OF THE DRAWINGS

The accompanying drawings, which are incorporated herein and form a part of the specification, illustrate the embodiments and, together with the description, further serve to explain the principles of the embodiments and to enable a person skilled in the relevant art(s) to make and use the embodiments.

FIG. 1 is a schematic diagram of an MRI system for capturing anatomic images of a patient and transmitting physiological data of a patient.

FIG. 2 is a schematic diagram of an electrocardiograph cable, according to some embodiments of the present disclosure, used in the MRI system of FIG. 1.

FIG. 3A is a perspective view of an electrically conductive wire and an electrically insulating core of a lead wire used in the electrocardiograph cable of FIG. 2 according to some embodiments of the present disclosure.

FIG. 3B is an enlarged detailed view of an electrically conductive wire and an electrically insulating core of a lead wire used in the electrocardiograph cable of FIG. 2 taken along broken line 3-3 of FIG. 3A according to some embodiments of the present disclosure.

FIG. 4A is a perspective view of an electrically conductive wire and an electrically insulating core of a lead wire used in the electrocardiograph cable of FIG. 2 according to some embodiments of the present disclosure.

FIG. 4B is an enlarged detailed view of an electrically conductive wire and an electrically insulating core of a lead wire used in the electrocardiograph cable of FIG. 2 taken along broken line 4-4 of FIG. 4A according to some embodiments of the present disclosure.

FIG. 5 is a table listing material and dimensional parameters of lead wires according to some embodiments of the present disclosure.

FIG. 6 is a block diagram showing a method of controlling surface temperature of a lead wire according to an embodiment of the present disclosure.

FIG. 7 is a cross-sectional view of electrocardiograph cable of FIG. 2 taken along broken line 8-8 of FIG. 2 according to some embodiments of the present disclosure.

The features and advantages of the embodiments will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

Embodiments of the present disclosure are described in detail with reference to embodiments thereof as illustrated in the accompanying drawings. References to “one embodiment,” “an embodiment,” “some embodiments,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following examples are illustrative, but not limiting, of the present embodiments. Other suitable modifications and adaptations of the variety of conditions and parameters normally encountered in the field, and which would be apparent to those skilled in the art, are within the spirit and scope of the disclosure.

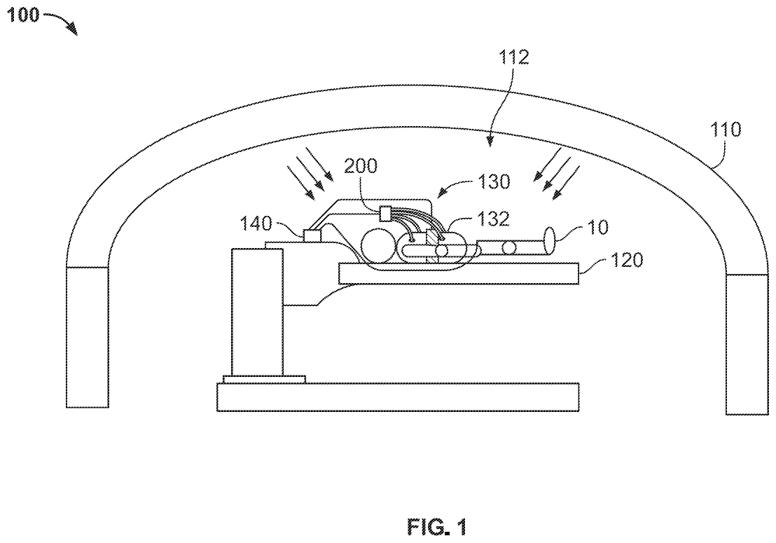

The present disclosure presents an electrocardiograph cable that is configured to reduce the risk of overheating that is commonly induced by pulsating RF waves applied by an MRI scanner in an MRI system, such as, for example, MRI system 100 shown schematically in FIG. 1. According to the embodiments described in more detail below, the electrocardiograph cable includes a plurality of lead wires for conducting electrocardiogram signals from monitoring electrodes. Each lead wire includes an adequate quantity of surface area and volume of conductive material to increase the heat transfer rate of the lead wire, while still providing a sufficient amount of resistance distributed along the lead wire to minimize the generation of eddy currents that is commonly induced by pulsating RF waves emitted during an MRI procedure.

MRI system 100 may include any device suitable for capturing anatomic images of a patient 10 and monitoring the physiological state of patient 10. For example, MRI system 100 may include an MRI scanner 110 configured to examine patient 10 at an observation area 112. MRI system 100 may include a patient bed 120 configured to displace patient 10 through observation area 112 of MRI scanner 110. MRI system 100 may include a monitoring apparatus 130 configured to monitor the physiological state of patient 10. MRI system 100 may include a transmitter 140 configured to transmit the monitored physiological parameters of patient 10 to a recording device during an imaging session.

MRI Scanner 110 may include any suitable component for generating magnetic fields and applying RF pulses into observation area 112 to capture anatomic images of patient 10. For example, MRI scanner 110 may include a magnet, such as a superconducting magnet, configured to generate a static magnetic field having a magnetic flux density of at least 1.5 Tesla (e.g., a magnetic flux density of 3.0 Tesla) into observation area 112. MRI scanner 110 may include a RF antenna configured to apply RF pulses, such as a gradient echo sequence, into observation area 112 for exciting atomic nuclei of the body of patient 10. The repetition rate for the RF pulses may range from about 10 Hz to 5 KHz. The RF pulses may be excited with 50 kW of RF power to create a magnetic field strength of at least 1500 V/M. MRI scanner 110 may include gradient coils configured to generate gradients in the magnetic field, such as, for example, a linear variation in the magnetic field. The magnetic field gradients may be used for selective slice excitation and for phase and frequency encoding of measurement signals.

Monitoring apparatus 130 may be configured to detect one or more physiological signals, such as, for example, electrocardiogram signals, respiration signals, and/or plethysmographic signals of patient 10. These signals may be used as reference values for the captured images by correlating the physiological signals with a respective image slice. For example, in cardiac imaging, accurately detecting the peak of the “R” wave of an electrocardiogram signal may ensure that each image slice is taken when the heart is in the same relative position.

In some embodiments, monitoring apparatus 130 may include a set of monitoring electrodes 132 attached to the patient's body (e.g., at the patient's torso) and configured to detect the electrocardiogram signals of patient 10. In some embodiments, electrodes 132 may be comprised of a carbon fiber material. Monitoring apparatus 130 may include an electrocardiograph cable 200 coupled to electrodes 132. Electrocardiograph cable 200 may be configured to conduct the patient's electrocardiogram signals detected by electrodes 132 to transmitter 140 or any other suitable patient monitor device, thereby allowing the patient's electrocardiogram signals to be tracked during the MRI procedure.

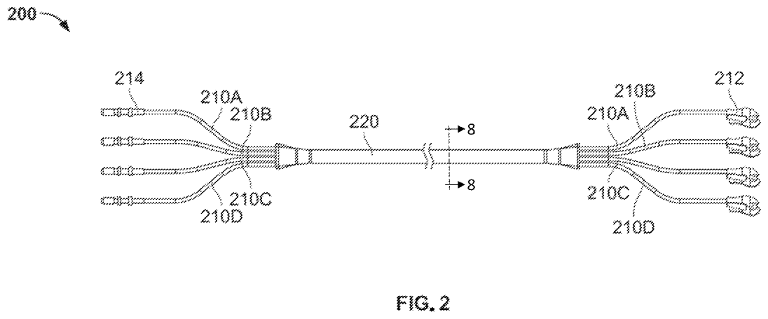

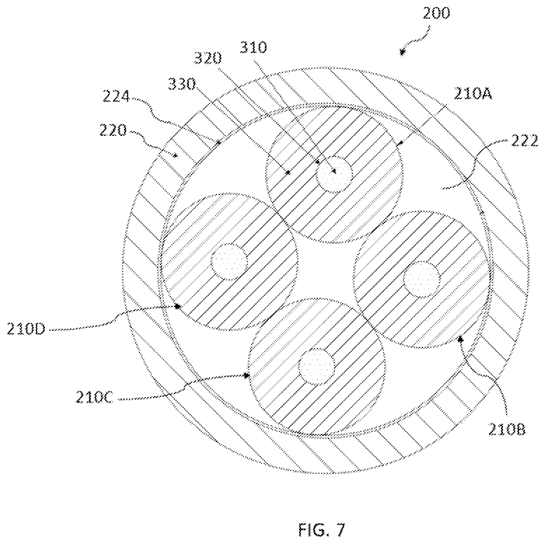

FIG. 2 shows a schematic diagram of electrocardiograph cable 200, according to some embodiments, for conducting the electrocardiogram signals from monitoring electrodes 132 to a monitoring device (e.g., transmitter 140). Electrocardiograph cable 200 is configured to reduce heating commonly induced by pulsating RF waves applied during an MRI session to prevent patient burns. Electrocardiograph cable 200 may include a plurality of lead wires 210A-D, with each lead wire 210A-D coupled to a respective monitoring electrode 132 and a monitoring device, such as transmitter 140. Electrocardiograph cable 200 may include a cable jacket 220 harnessing the plurality of lead wires 210A-D. For example, as shown in FIG. 7, each of lead wires 210A-D may extend through an internal passage 222 of cable jacket 220. In some embodiments, the plurality of lead wires 210A-D may be twisted together along the internal passage of the cable jacket 220. In some embodiments, the plurality of lead wires 210A-D may extend parallel with respect to each other along the internal passage of cable jacket 220.

In some embodiments, cable jacket 220 may be comprised of an electrically insulating material, such as, for example, polyvinylchloride, polyethylene, chlorinated polyethylene, ethylene propylene diene monomer, nitrile rubber, or combinations thereof. In some embodiments, cable jacket 220 may be comprised of a thermoplastic elastomer (TPE), such as for example, a Teknor Apex Medalist® Medical Grade TPE (e.g., Teknor Apex Medalist® MD-585). Cable jacket 220 may be formed by any method suitable for harnessing the lead wires 210A-D together, such as, for example, blow molding, injection molding, compression molding, and thermoforming. As shown in FIG. 7, in some embodiments, cable jacket 220 may include a paper wrap liner 224 disposed along the interior surface of cable jacket 220.

As shown in FIG. 2, lead wires 210A-D may each include a first end segment 212 configured to be coupled to a monitoring electrode, such as electrode 132. For example, in some embodiments, first end segment 212 may include an electrode connector that is configured to clamp electrode 132. Lead wires 210A-D may each include a second end segment 214 configured to be coupled to a monitoring device, such as transmitter 140 or a patient monitor. In some embodiments, second end segment 214 may include a metal terminal pin configured to be inserted into transmitter 140 or a patient monitor.

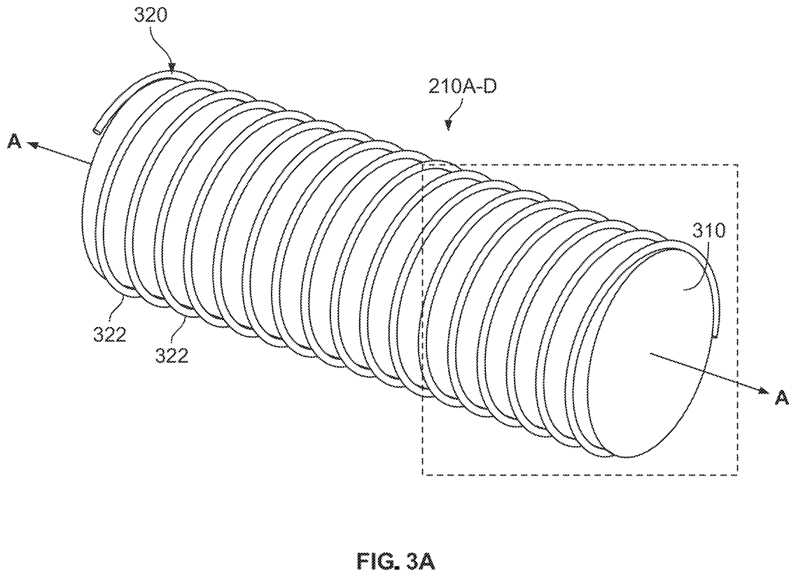

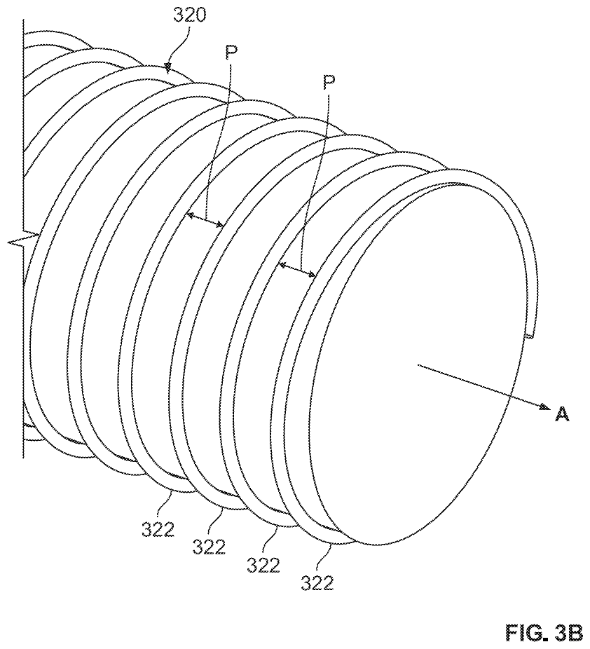

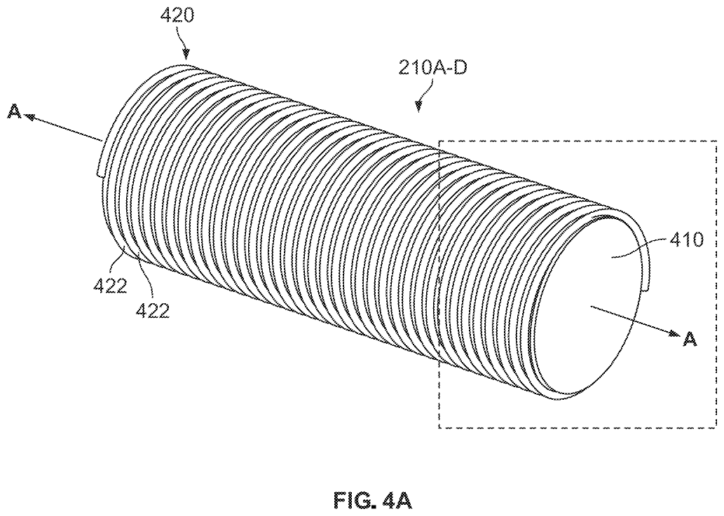

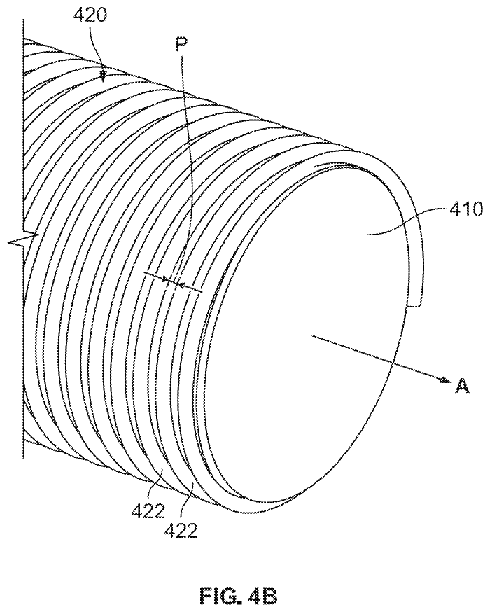

FIGS. 3A-4B show a perspective view of the interior design of lead wires 210A-D according to some embodiments. The interior design of lead wires 210A-D provides tight control of electrical resistance per unit length while increasing the lead wire's heat capacity to reduce the quantity of heat that is typically induced by RF waves applied during an MRI session. To reduce RF induced heating, the interior design of lead wires 210A-D is configured to increase the heat transfer rate from the interior conductive material to the exterior surface of lead wires 210A-D and minimize the generation of eddy currents in the conductive material of lead wires 210A-D.

Lead wires 210A-D may each include an electrically insulating core 310, 410; an electrically conductive wire 320, 420; and an outer electrically insulating sleeve 330.

Electrically insulating core 310, 410 may extend from first end segment 212 to second end segment 214 of lead wire 210A-D. Electrically insulating core 310, 410 may be comprised of any material suitable for inhibiting conduction of a current while providing a suitable combination of strength and flexibility. For example, in some embodiments, electrically insulating core 310, 410 may be comprised of a glass fiber-based material. Electrically insulating core 310, 410 may include a diameter in a range from about 0.20 inches to about 0.40 inches, such, as for example, a diameter in a range from about 0.25 inches to about 0.35 inches. These ranges of core diameter allow insulating core 310, 410 to have a sufficient amount of exterior surface area to receive additional conducting material, such as an electrically conductive wire, while providing lead wires 210A-D the flexibility needed for reaching electrodes 132 and monitoring devices. In some embodiments, electrically insulating core 310, 410 may include glass fiber core having a diameter of about 0.034 inches. In some embodiments, electrically insulating core 310, 410 may include a glass fiber core having a diameter of about 0.025 inches.

Electrically conductive wire 320, 420 may be helically wound around electrically insulating core 310, 410. The electrically conductive wire 320, 420 may be comprised of any material suitable for conducting an electric current while including an adequate amount of resistance (e.g., at least 100 ohms per foot) to prevent the generation of eddy currents from RF waves applied by an MRI scanner. For example, in some embodiments, electrically conductive wire 320, 420 may include a metal alloy-based material having a resistance in a range from about 650 ohm-cir-mil/foot to about 850 ohm-cir-mil/foot. This range of resistance provides an adequate amount of conductivity to transmit a charge, such as an electrocardiogram signal from electrode 132, while having enough resistance to reduce the likelihood of generating eddy currents during an MRI session. In some embodiments, the metal alloy of electrically conductive wire 320, 420 may include a combination of nickel and chromium (e.g., Nichrome). Electrically conductive wire 320, 420 may be devoid of any ferrous materials such that electrically conductive wire 320, 420 is non-magnetic. In some embodiments, electrically conductive wire 320, 420 may be a resistance wire that is devoid of any iron.

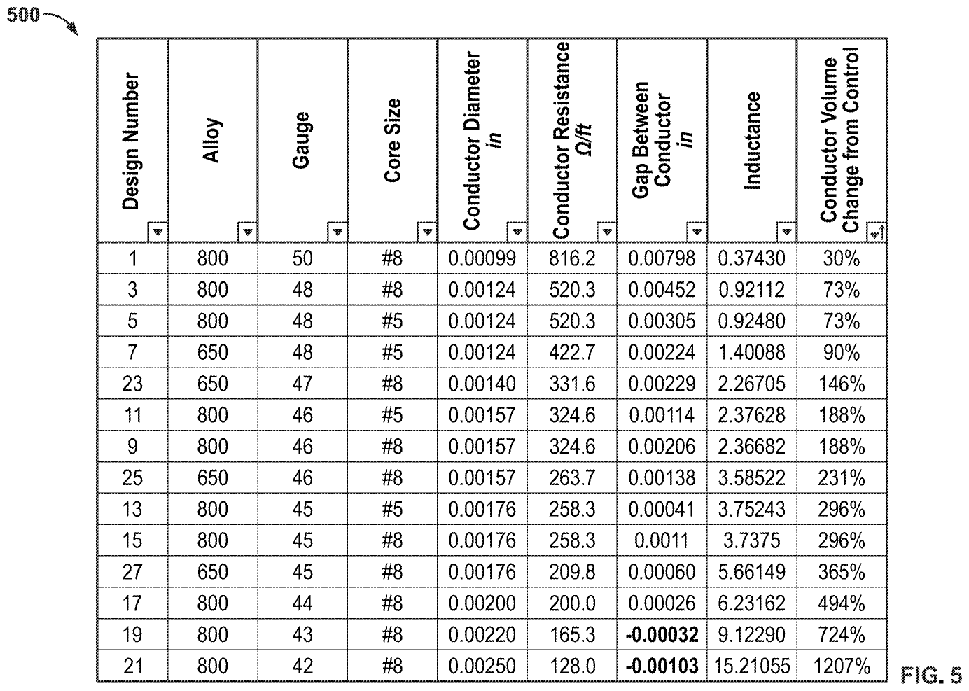

Electrically conductive wire 320, 420 may include a plurality of turns 322, 422 wound around electrically insulating core 310, 410 from first end segment 212 to second end segment 214 of lead wire 210A-D. The spatial arrangement of the plurality of turns 322, 422 of electrically conductive wire 320, 420 along a longitudinal axis A of the electrically insulating core 310, 410 is configured to increase the total conductor surface area of lead wire 210A-D, thereby increasing the heat capacity of lead wire 210A-D, ultimately improving the heat transfer performance of lead wires 210A-D. For example, a pitch P between adjacent turns 322, 422 of electrically conductive wire 320, 420 may remain substantially uniform along longitudinal axis A of electrically insulating core 310, 410 such that there is equal spacing between each pair of adjacent turns 322, 422 along electrically insulating core 310, 410. Pitch P between adjacent turns 322, 422 of electrically conductive wire 320, 420 may range from about 0.0020 inches to about 0.0001 inches, such as for example, a pitch P ranging from about 0.00138 inches (e.g. design 25 in Table 500 of FIG. 5) to about 0.00026 inches (e.g. design 17 in Table 500 of FIG. 5). For example, in some embodiments, pitch P between adjacent turns 322, 422 of electrically conductive wire 320, 420 may be about 0.0011 inches (e.g. Design 15 in Table 500 of FIG. 5). Pitch P between adjacent turns 322, 422 of electrically conductive wire 320, 420 may be reduced to the smaller values of this range (e.g., from about 0.0011 inches to 0.00026 inches) when the electrically insulating core 310, 410 has a larger a diameter (e.g., greater than about 0.030 inches). In the context of the present disclosure, pitch P corresponds to the distance measured from one point (e.g., radial center or edge) on one wire turn to a corresponding point (e.g., radial center or edge) of an adjacent wire turn, as shown in FIGS. 3B and 4B. The total number of turns 322, 422 of electrically conductive wire 320, 420 provide a distributed resistance of about 10,000 ohms/foot along longitudinal axis A of electrically insulating core 310, 410.

This range of pitches P between adjacent turns 322, 422 of electrically conductive wire 320, 420 provides an adequate amount of conductive surface area within the interior of lead wires 210A-D, thereby increasing the heat transfer rate from electrically conductive wire 320, 420 toward the exterior of lead wire 210A-D, while also ensuring that is a sufficient spacing between adjacent turns 322, 422 to avoid unintended contact by turns 322, 422 and to ease manufacturing of lead wires 210A-D. The heat transfer rate of electrically conductive wire 320, 420 achieved by this range of pitch P between adjacent turns 322, 422 ultimately prevents lead wires 210A-D from overheating when subjected to the operating conditions of a typical MRI session, such as a time-varying magnetic field having a magnetic flux density of least 3 Tesla with pulses of RF waves generated with 50 kW of RF power.

Electrically conductive wire 320, 420 may include a diameter ranging from about 0.0012 inches to about 0.0030 inches, such as, for example, a diameter ranging from about 0.00176 inches (e.g., 45 gauge wire) to about 0.00250 inches (e.g., 42 gauge wire). In some embodiments, electrically conductive wire 320, 420 includes a 45 gauge metal-alloy wire having a diameter of about 0.00176 inches. This range of diameters for electrically conductive wire 320, 420 provides an adequate quantity of conductive material within the interior of lead wires 210A-D while still maintaining a fine configuration that reduces the likelihood of eddy currents generating in the conductive material. The diameter of electrically conductive wire 320, 420 may be reduced to smaller values of this range (e.g., 0.0012 inches to about 0.0018 inches) when electrically conductive wire 320, 420 has a greater number of turns, such as, for example, when pitch P between adjacent turns 322, 422 ranges from about 0.0011 inches to 0.00026 inches.

As shown in FIG. 7, electrically insulating sleeve 330 may cover electrically conductive wire 320, 420 wound around electrically insulating core 310, 410. Electrically insulating sleeve 330 may be comprised of any material suitable for resisting charge of electrically conductive wire 320, 420 and shielding the interior of lead wires 210A-D. In some embodiments, insulating sleeve 330 may include a polymer-based material, such as, for example, polyethylene, ethylene tetrafluoroethylene (ETFE), polyurethane, silicone resin, or any combination thereof. In some embodiments, insulating sleeve 330 may include an elastomer-based material, such as a TPE, a thermoset rubber, or a combination thereof, such as, for example, Santoprene™.

The diameter of electrically insulating core 310, 410 and the internal resistance, the diameter, and the pitch of electrically conductive wire 320, 420 are collectively configured to reduce heating induced by RF waves applied during an MRI session. The internal resistance, diameter, and pitch of electrically conductive wire 320, 420 increases the heat capacity of lead wires 210A-D, while reducing the likelihood of generating eddy currents that is typically induced in conventional lead wires during an MRI session. Accordingly, each of the lead wires 210A-D is configured to maintain an outer surface of the electrically insulating sleeve at a temperature that does not burn or cause harm to patient 10 (e.g., a temperature of about 26° C. or less) when subjected to MRI operating conditions, such as a time-varying magnetic field having a magnetic flux density of at least 3 Tesla and a pulsating RF series of waves generated by 50 kW of RF power. That is, the internal resistance, the diameter, and the pitch of electrically conductive wire 320, 420 collectively prevent the insulating sleeve temperature from rising significantly above the ambient temperature of the medical environment during MRI operating conditions, thereby ensuring the safety of the patient.

FIG. 5 shows a Table 500 listing various embodiments of lead wires that were tested during the development of an electrocardiograph cable 200. During testing, the lead wires listed in Table 500 were subjected to operating conditions of a typical MRI session with other conventional lead wires to compare the thermal performance of the lead wires with the conventional lead wires. For example, during one scanning session of the test procedure, a set of newly developed lead wires and a set of conventional lead wires were disposed side-by-side in a bay of an MRI scanner and subjected to a magnetic field having a magnetic flux density of 3 Tesla and RF waves pulsating at a maximum operating power (e.g., 50 kW) of the MRI scanner for a predetermined time period. After completing the scanning session, temperature measurements of the newly developed lead wires and the conventional wires were taken and used to determine the surface temperatures of the insulating sleeves of the newly developed lead wires and the conventional wires.

Lead wire embodiments according to design numbers 11, 25, 13, 15, 27, 17, 19, and 21 of Table 500 each included wire internal resistances, core diameters, wire diameters, and wire turn pitches that meet the ranges described herein. By including the particular resistances, diameters, and pitches indicated in Table 500, the lead wire embodiments according to design numbers 11, 25, 13, 15, 27, and 17 exhibited improved heat transfer performance that maintained sleeve surface temperatures below the sleeve surface temperatures of other conventional lead wires during the test procedure described herein. Lead wire embodiments according to design numbers 13, 15, 27, and 17 yielded improved thermal performance (e.g., increased heat transfer rate and heat capacity) while still providing sufficient resistance to prevent the generation of eddy currents.

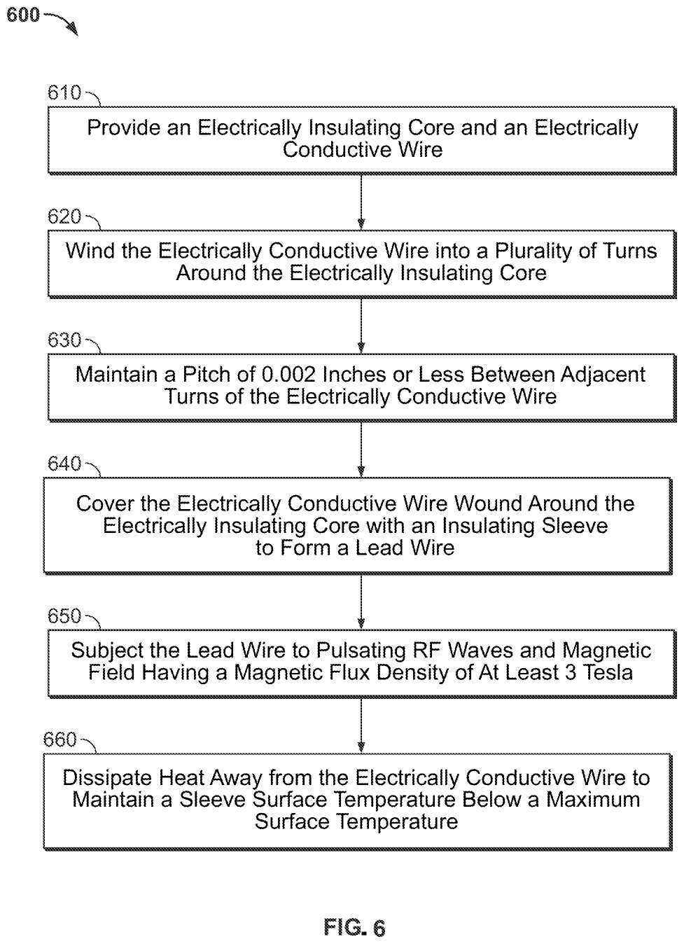

FIG. 6 shows an example block diagram illustrating a method 600, according to an embodiment, for controlling a temperature of lead wire 210A-D in electrocardiograph cable 200 during an MRI procedure.

Method 600 may include a step 610 of providing electrically insulating core 310, 410 with a diameter in a range from about 0.250 inches to about 0.035 inches and providing electrically conductive wire 320, 420 with a resistance in a range from 650 ohm-cir-mil/foot to about 850 ohm-cir-mil/foot. In some embodiments, step 610 may include providing a glass fiber core with a diameter of about 0.034 inches and an electrically conductive wire with a resistance of about 800 ohm-cir-mil/foot.

Method 600 may include a step 620 of winding electrically conductive wire 320, 420 into a plurality of turns 322, 422 around electrically insulating core 310, 410 from first end segment 212 of lead wire 210A-D to second end segment 214 of lead wire 210A-D. In some embodiments, step 620 may include winding a total number of turns 322, 422 that provides a distributed resistance of 10,000 ohms/foot along lead wire 210A-D.

Method 600 may include a step 630 of maintaining a pitch in a range from about 0.0020 inches to about 0.0001 inches between adjacent turns 322, 422 of electrically conductive wire 320, 420 uniformly along longitudinal axis A of electrically insulating core 310, 410. In some embodiments, step 630 may include maintaining a pitch of about 0.0011 inches between adjacent turns 322, 422 of electrically conductive wire 320, 420.

Method 600 may include a step 640 of covering electrically conductive wire 320, 420 wound around electrically insulating core 310, 410 with the electrically insulating sleeve of lead wire 210A-D. In some embodiments, step 640 may include using an elastomer-based material to cover electrically conductive wire 320, 420 wound around electrically insulating core 310, 410.

Method 600 may include a step 650 of subjecting lead wire 210A-D to pulsating RF waves and a time-varying magnetic field having a magnetic flux density in a range from about 1 Tesla to about 12 Tesla, such as for example, a time-varying field having a magnetic flux density in a range from 3 Tesla to 5 Tesla. In some embodiments, step 650 may include connecting lead wire 210A-D to a monitoring electrode coupled to patient 10 who is disposed in observation area 112 of MRI scanner 110. In some embodiments, step 650 may include using 50 kW of RF power by MRI scanner 110 to generate the pulsating RF waves.

Method 600 may include a step 660 of dissipating heat away from electrically conductive wire 320, 420 to maintain sleeve surface temperature of lead wire 210A-D below a maximum surface temperature. In some embodiments, step 660 may include a maximum surface temperature ranging from about 15° C. to about 26° C., such as, for example 26° C., to prevent lead wire 210A-D from causing burns to a skin of a patient. In some embodiments, step 660 may include controlling rise of the sleeve surface temperature above the ambient temperature of the medical environment (e.g., 20° C.) to be within a predetermined range. For example, if the ambient temperature of the medical environment is 20° C., conductive wire 320, 420 may be configured to dissipate enough heat during the operating conditions set in step 650 to maintain a maximum temperature rise of the sleeve surface in a range from about 2° C. to about 6° C. above the ambient temperature of the medical environment.

It is to be appreciated that the Detailed Description section, and not the Brief Summary and Abstract sections, is intended to be used to interpret the claims. The Summary and Abstract sections may set forth one or more but not all exemplary embodiments as contemplated by the inventors, and thus, are not intended to limit the present embodiments and the appended claims in any way.

The foregoing description of the specific embodiments will so fully reveal the general nature of the inventions that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the claims and their equivalents.

What is claimed is:

1. An electrocardiograph cable for reducing heat induced by pulsating radio frequency waves applied by a magnetic resonance imaging (MRI) scanner, comprising:
a cable jacket; and
a plurality of lead wires extending through the cable jacket, wherein each of the plurality of lead wires comprises:
a first end segment configured to be coupled to a monitoring electrode,
a second end segment configured to be coupled to a monitoring device,
an electrically insulating core extending from the first end segment to the second end segment,
an electrically conductive wire having a plurality of turns wound around the electrically insulating core from the first end segment to the second end segment, and
an electrically insulating sleeve covering the electrically conductive wire wound around the electrically insulating core;
wherein a pitch between adjacent turns of the electrically conductive wire remains substantially uniform along a longitudinal axis of the electrically insulating core, and the pitch between adjacent turns of the electrically conductive wire is in a range from about 0.0020 inches to about 0.0001 inches.

2. The electrocardiograph cable of claim 1, wherein the electrically conductive wire comprises a metal alloy-based material having a resistance in a range from about 650 ohm-cir-mil/foot to about 850 ohm-cir-mil/foot.

3. The electrocardiograph cable of claim 3, wherein the metal alloy-based material of the electrically conductive wire is non-magnetic.

4. The electrocardiograph cable of claim 1, wherein the electrically conductive wire comprises a diameter in a range from about 0.00176 inches to about 0.00250 inches.

5. The electrocardiograph cable of claim 1, wherein the electrically insulating core comprises a glass fiber-based material.

6. The electrocardiograph cable of claim 1, wherein the electrically insulating core comprises a diameter in a range from about 0.250 inches to about 0.035 inches.

7. The electrocardiograph cable of claim 1, wherein each of the lead wires is configured to maintain an outer surface of the electrically insulating sleeve at a temperature in a range from about 26° C. to about 15° C. when subjected to a time-varying magnetic field having a magnetic flux density in a range from about 3 Tesla to about 10 Tesla.

8. The electrocardiograph cable of claim 1, wherein the electrically insulating sleeve comprises an elastomer-based material.

9. The electrocardiograph cable of claim 1, wherein each of the lead wires has a distributed resistance of about 10,000 ohms/foot.

10. The electrocardiograph cable of claim 1, wherein the plurality of lead wires are twisted together along an internal passage of the cable jacket.

11. A lead wire in an electrocardiograph cable for reducing heat induced by pulsating radio frequency waves applied by a magnetic resonance imaging (MRI) scanner, comprising:
a first end segment configured to be coupled to a monitoring electrode;
a second end segment configured to be coupled to a monitoring device;
an electrically insulating core extending from the first end segment to the second end segment, wherein the electrically insulating core comprises a diameter in a range from about 0.250 inches to about 0.035 inches;
an electrically conductive wire having a plurality of turns wound around the electrically insulating core from the first end segment to the second end segment; and
an electrically insulating sleeve covering the electrically conductive wire wound around the electrically insulating core;
wherein the electrically conductive wire comprises a heat capacity configured to maintain an outer surface of the electrically insulating sleeve at a temperature in a range from about 26° C. to about 15° C. when subjected to a time-varying magnetic field having a magnetic flux density in a range from about 3 Tesla to about 10 Tesla.

12. The lead wire of claim 11, wherein a pitch between adjacent turns of the electrically conductive wire remains substantially uniform along a longitudinal axis of the electrically insulating core.

13. The lead wire of claim 12, wherein the pitch between adjacent turns of the electrically conductive wire is in a range from about 0.0020 inches to about 0.0001 inches.

14. The lead wire of claim 11, wherein the electrically conductive wire comprises a metal alloy-based material having a resistance in a range from about 650 ohm-cir-mil/foot to about 850 ohm-cir-mil/foot.

15. The lead wire of claim 14, wherein the metal alloy-based material of the electrically conductive wire is non-magnetic.

16. The lead wire of claim 11, wherein the electrically conductive wire comprises a diameter in a range from about 0.00176 inches to about 0.00250 inches.

17. The lead wire of claim 11, wherein the electrically insulating core comprises a glass fiber-based material.

18. The lead wire of claim 11, wherein the electrically insulating sleeve is comprises an elastomer-based material.

19. The lead wire of claim 11 further comprising a distributed resistance of about 10,000 ohms/foot

20. A method for controlling a temperature of a lead wire in an electrocardiograph cable during a magnetic resonance imaging (MRI) procedure, comprising:
providing an electrically insulating core of the lead wire with a diameter in a range from about 0.250 inches to about 0.035 inches;
providing an electrically conductive wire of the lead wire with a resistance in a range from 650 ohm-cir-mil/foot to about 850 ohm-cir-mil/foot;
winding the electrically conductive wire into a plurality of turns around the electrically insulating core from a first end segment of the lead wire to a second end segment of the lead wire;
maintaining a pitch in a range from about 0.0020 inches to about 0.0001 inches between adjacent turns of the electrically conductive wire uniformly along a longitudinal axis of the electrically insulating core;
covering the electrically conductive wire wound around the electrically insulating core with an electrically insulating sleeve of the lead wire;
subjecting the lead wire to pulsating radio frequency waves and a time-varying magnetic field having a magnetic flux density in a range from about 3 Tesla to about 10 Tesla; and
dissipating heat away from the electrically conductive wire to maintain an outer surface of the electrically insulating sleeve at a temperature in a range from about 26° C. to about 15° C.
